(19) United States
(12) Patent Application Publication
(10) Pub. No.: US 2024/0210417 A1
(43) Pub. Date: Jun. 27, 2024

(54) KIT AND METHOD FOR SCREENING OF ANTIBODY USING GPCR-EMBEDDED NANODISCS

(71) Applicants: RECEPTECH INC., Seoul (KR); SEOUL NATIONAL UNIVERSITY R&DB FOUNDATION, Seoul (KR)

(72) Inventors: Hwi Jin KO, Seoul (KR); Tai Hyun PARK, Seoul (KR); Soo KIM, Suwon-si (KR)

(73) Assignees: RECEPTECH INC., Seoul (KR); SEOUL NATIONAL UNIVERSITY R&DB FOUNDATION, Seoul (KR)

(21) Appl. No.: 18/556,120

(22) PCT Filed: Nov. 8, 2021
(86) PCT No.: PCT/KR2021/016144

§ 371 Date: Oct. 19, 2023

(30) Foreign Application Priority Data
Apr. 19, 2021 (KR) 10-2021-0050481

(51) Int. Cl. G01N 33/68; G01N 33/531

(52) U.S. Cl. CPC G01N 33/6854; G01N 33/531; G01N 33/6872; G01N 2333/726

(57) ABSTRACT

The present invention relates to an antibody screening kit equipped with a G protein-coupled receptor (GPCR)-embedded nanodisc, and a method for screening an antibody drug, using same.

### TECHNICAL FIELD

The present disclosure relates to an antibody screening kit equipped with GPCR-embedded nanodiscs, a method for manufacturing the kit, and a method for screening antibody drugs using the kit.

### BACKGROUND ART

G protein-coupled receptors (GPCRs) play an important role in cellular responses in the human body. Therefore, as many as approximately one-third to one-half of currently prescribed small-molecule synthetic drugs target GPCRs, underscoring the immense significance of GPCRs as pharmaceutical targets. While numerous small-molecule drugs targeting GPCRs are in development and use, only two antibody drugs targeting GPCRs have been approved by the FDA to date. The global pharmaceutical market is witnessing an increasing share of biopharmaceuticals. Among them, antibody drugs dominate the top sales rankings and are on a growing trend. A primary reason for the underdevelopment of antibody drugs targeting GPCRs is the difficulty in mass-producing GPCRs with a properly formed structure, which serve as antigens. Additionally, there is a lack of an efficient antibody screening systems utilizing GPCRs.

### PRIOR ART DOCUMENT

### Patent Literature

Korean Patent No. 10-1994279 A

### DISCLOSURE OF INVENTION

### Technical Problem

Leading to the present disclosure, intensive and thorough research conducted by the present inventors with the aim of overcoming problems encountered in the related art resulted in the development of a novel GPCR-embedded nanodisc, produced from GPCR E. coli, which mimics the original receptor structure and is stable in water and air environments. GPCR, which is a target for disease treatment, is inserted into a nanodisc in a stable form with its original function, and an antibody screening kit equipped with the nanodisc thus manufactured was found to specifically identify only effective antibodies among various types of antibodies.

The present disclosure also aims to provide a method for screening an antibody drug using the antibody screening kit.

### Solution to Problem

The present disclosure provides an antibody screening kit equipped with a G protein-coupled receptor (GPCR)-embedded nanodisc.

In addition, the present disclosure provides a method for screening an antibody drug, using the antibody screening kit.

### Advantageous Effects of Invention

According to the present disclosure, GPCR, which is a target for disease treatment, is inserted into a nanodisc in a stable form having an original function, and an antibody screening kit equipped with the resulting nanodisc can be manufactured. Since the kit can specifically select only effective antibodies from among various types of antibodies, it can be effectively used in the development of new antibody drugs.

### BRIEF DESCRIPTION OF DRAWINGS

FIG. 1 is an image of Western blots for CCR4 protein, a type of GPCRs, expressed in E. coli according to an embodiment of the present disclosure.

FIG. 2 is an image of the SDS-PAGE results for the membrane scaffold protein (MSP1E3D1) purified according to an embodiment of the present disclosure.

FIG. 3 is a size exclusion chromatograph for CCR4 nanodiscs purified according to an embodiment.

FIG. 4 is a graph of CCR4 nanodisc sizes as measured by DLS according to an embodiment of the present disclosure.

FIG. 5 is a plot of intensities of mogamulizumab measured against the concentration of the CCR4 nanodiscs immobilized according to an embodiment of the present disclosure.

FIG. 6 is a plot of intensities of mogamulizumab measured against the concentration of mogamulizumab.

FIG. 7 is a plot illustrating the selective detection of mogamulizumab from an antibody mixture according to an embodiment of the present disclosure.

### BEST MODE FOR CARRYING OUT THE INVENTION

Below, a detailed description will be given of the present disclosure.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It will be further understood that the terms “include”, “comprises” “including” and/or “comprising,” when used in this specification, specify the presence of stated features, integers, steps, operations, elements, and/or components, but do not preclude the presence or addition of one or more other features, integers, steps, operations, elements, components, and/or groups thereof.

An aspect of the present disclosure provides an antibody screening kit equipped with a G protein-coupled receptor (GPCR)-embedded nanodisc.

The G protein-coupled receptor (GPCR) may be a chemokine receptor, preferably a CC chemokine receptor, and more preferably a CC chemokine receptor type 4 (CCR4).

The CC chemokine receptor is an integral membrane protein that specifically binds and responds to CC chemokines. Chemokines consist of approximately 70 to 130 amino acids with four cysteine residues connected by disulfide bonds, where the first two consecutive cysteine residues are referred to as CC chemokines. Chemokines play a pivotal role in the migration of white blood cells to sites of inflammation or immune responses. They are secreted from white blood cells or tissue cells under basal conditions or specific stimuli and typically act locally in a paracrine or autocrine manner, similar to cytokines. Chemokines can be secreted from a variety of cells, including blood and tissue cells. Chemokines and their receptors are involved in various processes such as inflammation, immune regulation, virus penetration suppression or acceptance, hematopoiesis regulation, angiogenesis control, lymphatic tissue development, wound healing, cancer metastasis, and antitumor actions. CC chemokines generally lack selectivity and attract various types of white blood cells, for instance, monocytes, eosinophils, basophils, T-lymphocytes, and natural killer cells. CC chemokines such human monocyte as chemoattractant proteins 1-3 (MCP-1, MCP-2, and MCP-3), RANTES (Regulated on Activation, Normal T Expressed and Secreted), and macrophage inflammatory proteins 1α and 1β (MIP-1α and MIP-1β) are known to be primary and active chemoattractants for monocytes or lymphocytes. The CC chemokine receptor type 4 (CCR4) is a predominant chemokine receptor expressed in human Tregs. CCR4 can bind with the ligands CCL17 and CCL22. When a significant amount of CCL17 is secreted at inflammation sites, the CCR4-expressing (CCR4+) Tregs that recognize this condition migrate to the site of inflammation, potentially mitigating the inflammatory response.

According to an embodiment of the present disclosure, the antibody screening kit equipped with a CC chemokine receptor type 4 (CCR4)-embedded nanodisc may effectively screen a CCR4 antibody.

By way of example, the CCR4 antibody may be mogamulizumab. Mogamulizumab, which is a CCR4 monoclonal antibody, is active against various T-cell lymphomas.

The G protein-coupled receptor (GPCR)-embedded nanodisc may be manufactured by a method including the steps of: a) producing and purifying G protein-coupled receptors (GPCR) in E. coli cells; b) producing and purifying membrane scaffold proteins in E. coli cells; and c) mixing and stirring lipids, the membrane scaffold proteins, and G protein-coupled receptors (GPCRs) in the 25 order thereof to assemble a nanodisc.

In the present disclosure, E. coli cells transformed with a G protein-coupled receptor (GPCR) gene are first cultured to a certain level. The cultured cells are allowed to overexpress the G protein-coupled receptor (GPCR) therein and then lysed to extracellularly release the G protein-coupled receptor (GPCR) overexpressed in particulate forms. Afterwards, the released G protein-coupled receptors (GPCRs) are solubilized using a surfactant, etc., separated and purified, and mixed with membrane scaffold proteins and lipids to reconstitute nanodiscs which mimic the original receptor structure and thus can be stable even in water and atmospheric environments.

In the step a) of producing G protein-coupled receptors (GPCRs), GPCRs are expressed as inclusion bodies within E. coli cells which are then lysed to extracellularly release the inclusion bodies of the protein, followed by solubilizing, purifying, and reconstituting the inclusion bodies back to the GPCR form. According to an embodiment of the invention, the step a) of producing G protein-coupled receptors (GPCRs) in E. coli cells may include: a1) culturing the E. coli transformed with G protein-coupled receptors (GPCRs); a2) overexpressing the G protein-coupled receptors (GPCRs); a3) lysing the E. coli to release the G protein-coupled receptors (GPCRs) outside the cell; and a4) solubilizing and purifying the G protein-coupled receptors (GPCRs).

The G protein-coupled receptors (GPCRs) produced in E. coli cells may be a chemokine receptor, preferably a CC chemokine receptor, and more preferably chemokine receptor type 4 (CCR4).

Any protein may be available as the membrane scaffold protein in step b) as long as it functions to surround a lipid-receptor composite. Preferable is membrane scaffold protein (MSP1E3D1).

The lipids may be selected from 1,2-dipalmitoyl-sn-glycero-3-phosphocholine (DPPC), 1,2-disteroylsn-glycero-3-phosphocholine (DSPC), L-a-phosphatidylcholine (HSPC), 1-palmitoyl-2-glutaroyl-sn-glycero-3-phosphocholine (PGPC), 1,2-dilauroyl-sn-glycero-3-phosphocholine (DLPC), 1,2-dimyristoyl-sn-glycero-3-phosphocholine (DMPC), 1,2-dioleyl-sn-glycero-3-phosphocholine (DOPC), and a mixture thereof.

In step c), the G protein-coupled receptors may be mixed at a molar ratio of 1:2 to 1:30 with the membrane scaffold proteins and at a molar ratio of 1:200 to 1:2500 with the lipids.

In an embodiment of the present disclosure, the molar ratio of G protein-coupled receptors:membrane scaffold proteins:lipids may be 1:20:2300.

Also, the present disclosure provides a nanodisc, manufactured by the manufacturing method of the present disclosure, has G protein-coupled receptors (GPCRs) embedded therein and is 10 nm in mean diameter and preferably 10 nm to 25 nm in mean diameter.

The nanodisc manufactured by the present disclosure is structured to have G protein-coupled receptors (GPCRs) surrounded by membrane scaffold proteins in E. coli cells.

The nanodisc manufactured by the present disclosure has the structure in which one type of G protein-coupled receptors (GPCRs) is embedded into a single nanodisc.

According to an embodiment of the present disclosure, the G protein-coupled receptor (GPCR)-embedded nanodiscs manufactured by the manufacturing method of the present disclosure were observed to have a mean particle size of 19.8 nm as measured by Dynamic Light Scattering (DLS).

Furthermore, the present disclosure provides a method for screening an antibody drug, using the antibody-screening kit.

The antibody drug is a pharmaceutical designed to specifically bind to an antigen protein responsible for a certain disease through an antigen-antibody reaction.

The antibody drug may include a chimeric monoclonal antibody, a humanized monoclonal antibody, a human monoclonal antibody, an antibody fragment, etc.

### MODE FOR CARRYING OUT THE INVENTION

A better understanding of the present disclosure may be obtained through the following examples, which are set forth to illustrate, but are not to be construed to limit, the present disclosure

### Example 1: Gene Cloning for Production of CCR4 in E. coli and Expression and Purification Thereof

A gene encoding CC chemokine receptor type 4 (CCR4), which is a membrane protein belonging to GPCRs, was amplified by PCR. In this regard, the gene was cloned into the pET-DEST42 vector so as to insert a His-tag into the C-terminus of CCR4 which would be thus readily immobilized to the bottom of the Ni-coated well. To express CCR4, Rosetta 2 E. coli was co-transformed with PET-DEST42/CCR4 and pBAD33.1/rraA vectors and then cultured at 37° C. in Luria-Bertani (LB) broth. The expression efficiency of CCR4 was improved by co-expressing rraA, known to increase expression efficiency of membrane proteins. After inoculation of the cells thereinto, the broth was added with 0.2% arabinose, an induce the expression accelerator, to preferentially expression of the rraA gene on pBAD33.1. Incubation was continued until an OD₆₀₀ of 0.5. When the OD₆₀₀ value reached 0.5, the pET-DEST42 vector was induced to overexpress CCR4 in the presence of 1 mM of isopropyl thiogalactoside (IPTG), used as an expression inducer for the vector.

Following an additional four hours of cell growth, the culture was centrifuged at 7000 g, 4° C. for 20 minutes and the cell pellet thus formed was resuspended in 2 mM EDTA in PBS (pH 8.0). The cells were lysed by sonication (5 seconds on/off, 5 minutes). The cell lysate was centrifuged at 12000 g, 4° C. for 20 minutes. The sonication and centrifugation were repeated under the same condition for the pellet. The pelleted cell lysate thus obtained was suspended in a solubilization buffer (0.1 M Tris-HCl, 20 mM Sodium dodecyl sulfate (SDS), 1 mM EDTA, 100 mM Dithiothreitol (DTT), pH 8.0) and solubilized overnight at 30° C. in a shaking incubator.

Solubilized samples were loaded into the membrane of 10 K MWCO dialysis cassette (Thermo Fisher Scientific, USA) and dialyzed overnight against dialysis buffer (0.1 M sodium phosphate, 10 mM SDS, pH 8.0). The dialyzed samples were filtered through 0.22 μm bottle top filter (Jetbiofil, Korea). Filtered proteins were purified using a HisTrap HP column and then dialyzed against HEPES buffer (20 mM HEPES-NaOH, 100 mM NaCl, 25 mM cholate) for storage using 10K MWCO dialysis cassette.

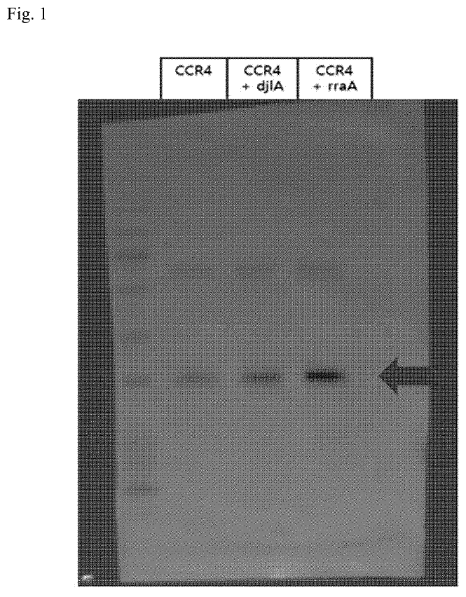

As shown in FIG. 1, the expression of CCR4 was identified by Western blot analysis and observed to increase with the co-expression of rraA.

The dialyzed proteins were stored at −80° C. until use.

### Example 2: Gene Cloning, Expression, and Purification of MSP1E3D1

MSP1E3D1, a membrane scaffold protein (MSP), was expressed. To this end, Rosetta 2 competent cells were transformed with pET-28a and incubated at 37° C. in Luria-Bertani (LB) broth. Incubation was continued until an OD₆₀₀ of 0.5. When the OD₆₀₀ value reached 0.5, the pET-28a vector was induced to overexpress MSP1E3D1 in the presence of 1 mM of isopropyl thiogalactoside (IPTG), used as an expression inducer for the vector. After being cultured overnight, the cell culture was centrifuged at 7000 g, 4° C. for 20 minutes and the cell pellet thus formed was resuspended in a MSP1E3D1-purifying buffer (Tris-HCl 20 mM, 0.5 M NaCl, 20 mM imidazole). The cells were lysed by sonication (5 seconds on/off, 5 minutes). The cell lysate was centrifuged at 12000 g, 4° C. for 20 minutes. The suspension sample following centrifugation was filtered through 0.22 μm bottle top filter (Jetbiofil, Korea).

Filtered proteins were purified using a HisTrap HP column and then dialyzed against HEPES buffer (20 mM HEPES-NaOH, 100 mM NaCl, 25 mM cholate) for storage using 10K MWCO dialysis cassette.

The filtered sample was purified through a HisTrap HP column using a MSP1E3D1-purifying buffer 2 (Tris-HCl 20 mM, 0.5 M NaCl, 350 mM imidazole) on the basis of the difference in binding affinity between the column and the protein, caused the by difference in imidazole concentration. Thereafter, the salt imidazole was removed using a HiTrap desalting column while the sample's buffer was changed to HEPES buffer 2 (20 mM HEPES-NaOH, 100 mM NaCl) for storage.

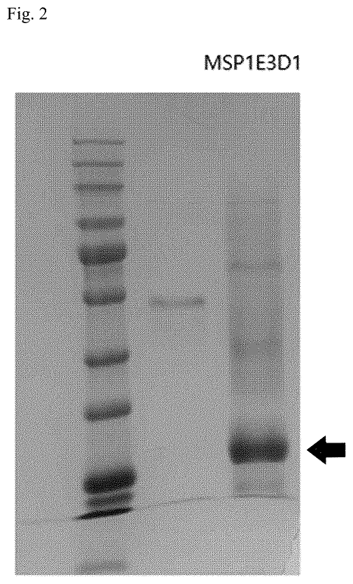

MSP1E3D1 was isolated by SDS-PAGE. FIG. 2 is an image of SDS-PAGE for isolating MSP1E3D1.

The MSP1E3D1 thus obtained was in the form of having a His-tag attached to the C-terminus thereof. In order to construct a nanodisc embedded with His-tag-labeled GPCR, MSP1E3D1 free of His-tag was employed. To this end, TEV protease was used to remove the His-tag from the C-terminus of MSP1E3D1.

### Example 3: Construction of CCR4-Embedded Nanodisc and Fabrication of Antibody Screening Kit Using Same

### Construction of CCR4-Embedded Nanodisc

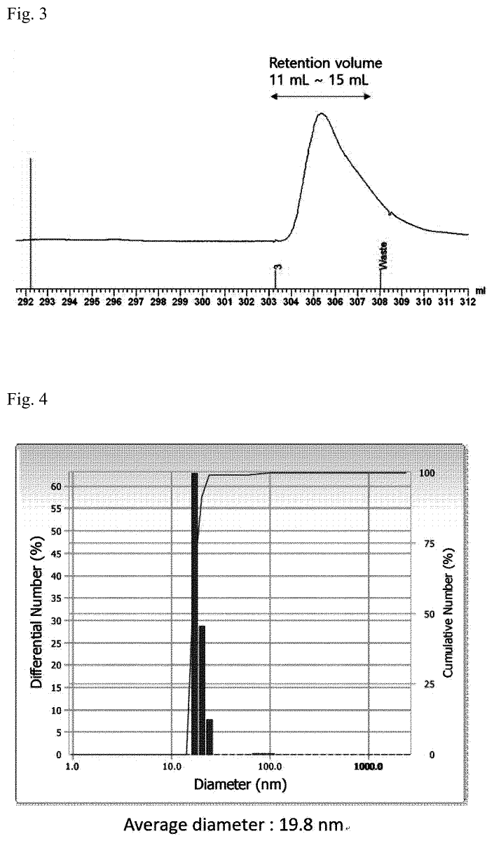

Nanodiscs were constructed using CCR4, the membrane scaffold protein (MSP1E3D1), and 1,2-dimyristoyl-sn-glycero-3-phosphocholine (DMPC). In brief, the three components DMPC, MSP, and CCR4 were mixed at the molar ratio of 1:20:2300 in that order and then incubated at 25° C., 170 rpm for 2 hours in a shaking incubator. Then, degassed Bio-Beads were added to the sample (0.6 g/ml (sample)) to remove the surfactant in the buffer, followed by stirring overnight in the same condition. Next, the sample was filtered through a 0.45 μm-filter to remove Bio-Beads. The filtered sample was applied to a HisTrap HP column to separate receptor-embedded nanodiscs which were then purified by finally size exclusion chromatography (Superdex 200 Increase 10/300 GE GL, Healthcare, USA) using 20 mM HEPES-NaOH and 100 mM NaCl buffer. As shown in FIG. 3, CCR4-embedded nanodiscs were separated and purified by size exclusion chromatography.

As seen in FIG. 4, the CCR4-embeded nanodiscs were observed to have a mean size of 19.8 nm as measured by DLS. The nanodiscs embedded with CCR4 having His-tag labeled at the C-terminus thereof were immobilized to nickel-coated 96-well plates (Thermo Fisher).

### Fabrication of Antibody Screening Kit

To determine the amount of nanodiscs to be immobilized, nanodiscs were added at various concentrations ranging from 2 nM to 256 nM.

Each well was added with 100 μL of the nanodiscs various concentrations quantitated by measuring with absorbance at 280 nm and incubated on a rocking table for 2 hours at room temperature. Any unbound nanodisc was removed by washing twice with 0.5% PBS-T. In addition, to prevent non-specific binding to the bottom of the well that may occur when using the kit of the present disclosure in the future, BSA solution (200 μL for each well) was used and incubated on a rocking table for 30 minutes. Each well was washed twice with 0.5% PBS-T. Thus, an antibody screening kit having CCR4-embedded nanodiscs immobilized thereto were fabricated.

### Experimental Example 1: Effectiveness of Antibody Screening Kit

One hundred μL of 3.35 nM mogamulizumab solution was added to each well and incubated for 1 hour on a rocking table. After two washes with 0.5% PBS-T, 100 μL of a secondary antibody (HRP-conjugated antibody, 1.66 nM) corresponding to the primary antibody (CCR4 antibody, mogamulizumab Kyowa (manufactured by Hakko Kirin)) was added to each well and incubated for 1 hour on a rocking table. After two washes with 0.5% PBS-T, each well was added with 100 μL of tetramethylbenzidine (TMB) substrate and incubated at 37° C. for 30 minutes in a light-tight condition, followed by reading absorbance at 450 nm on a microplate reader (TECAN, USA). The results are depicted in FIG. 5.

From FIG. 5, it can be observed that as the concentration of nanodiscs increases in ranges above 50 nM, the measurement intensity of the CCR4 antibody Mogamulizumab gradually increases. That is, at a CCR4 nanodisc concentration above 50 nM, Mogamulizumab was measured to exhibit a significant intensity, whereas there was no increase in absorbance for CCR5 antibody, a similar primary antibody. From this, it can be inferred that the CCR4 nanodiscs immobilized on the well bottom do not bind with the CCR5 antibody, and selectively bind only with the CCR4 antibody.

### Experimental Example 2: Effectiveness of Antibody Screening Kit

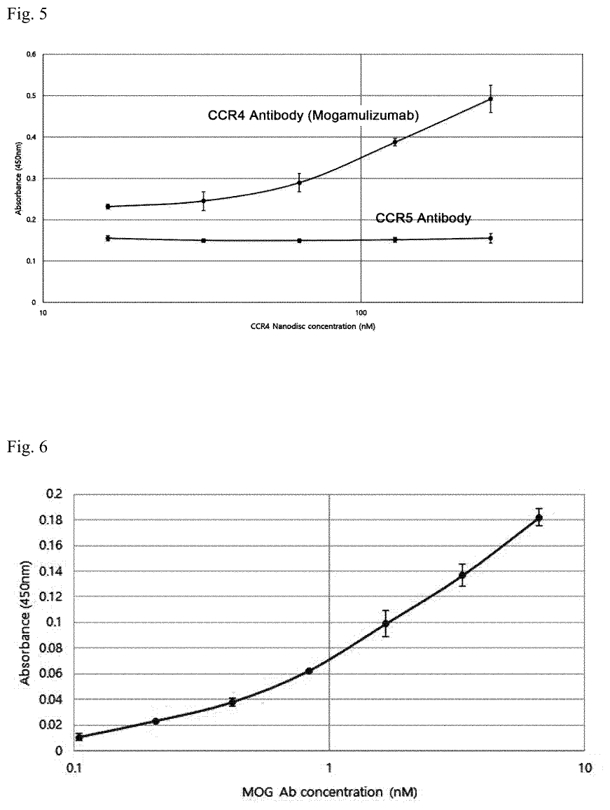

To validate the effectiveness of the antibody screening kit, absorbance was measured at varying concentrations of the CCR4 antibody mogamulizumab under the experimental condition using 128 nM CCR4 nanodiscs, 10.7 nM secondary antibody, and a non-specific reaction prevention solution (5% BSA). The results are presented in FIG. 6.

As seen in FIG. 6, the absorbance rose with the increasing of the concentration of mogamulizumab, indicating that the antibody screening kit is useful as a kit for screening mogamulizumab.

### Experimental Example 3: Reliability of Antibody Screening Kit

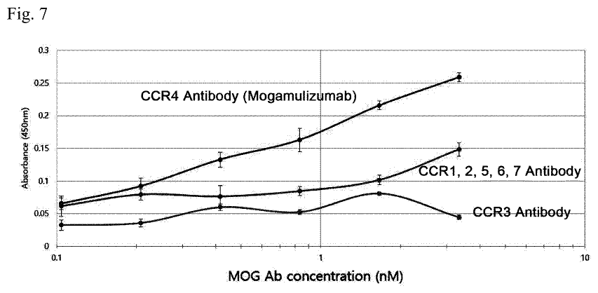

To verify whether the antibody screening kit can selectively screen a specific antibody from a mixture of antibodies, an experiment was conducted using a mixed solution containing 7 types of antibodies from the CCR series (CCR1-CCR7) under the following experimental conditions. The concentration of the six antibodies other than the target antibody, CCR4 antibody, was fixed at 3.35 nM each. The concentration of the CCR4 antibody was varied between 0.10 nM and 3.35 nM for the experiment. To determine whether the target antibody, CCR4 antibody, could be selectively detected even at concentrations lower than those of other antibodies, the experiment was performed at these concentrations, and the results are presented in FIG. 7.

### <Test Condition>

CCR4 nanodisc concentration: 128 nM, secondary Ab concentration: 10.7 nM, non-specific reaction prevention solution: 5% BSA
CCR1, 2, 5, 6, 7 Abs: each 3.35 nM, goat-derived Ab
CCR3 Ab: 3.35 nM, mouse-derived Ab
CCR4 Ab (mogamulizumab): 0.10 nM˜3.35 nM, human Ab
Secondary Ab to CCR1, 2, 5, 6, and 7 Abs: Rabbit anti-goat IgG
Secondary Ab to CCR3 Ab: Goat anti-mouse IgG
Secondary Ab to CCR4 Ab: Goat anti-human IgG

From data of FIG. 7, it is understood that selection can be made only of the target antibody CCR4 Ab (mogamulizumab) even from a mixture of CCR1 to CCR7.

1. An antibody screening kit, being equipped with a G protein-coupled receptor (GPCR)-embedded nanodisc.

2. The antibody screening kit of claim 1, wherein the G protein-coupled receptor (GPCR) is a chemokine receptor.

3. The antibody screening kit of claim 1, wherein the G protein-coupled receptor (GPCR) is a CC chemokine receptor.

4. The antibody screening kit of claim 1, wherein the G protein-coupled receptor (GPCR) is a CC chemokine receptor type 4.

5. The antibody screening kit of claim 1, wherein the G protein-coupled receptor (GPCR)-embedded nanodisc is manufactured by a manufacturing method comprising the steps of:
a) producing and purifying G protein-coupled receptors (GPCR) in E. coli cells;
b) producing and purifying membrane scaffold proteins in E. coli cells; and
c) mixing and stirring lipids, the membrane scaffold proteins, and G protein-coupled receptors (GPCRs) in the order thereof to assemble a nanodisc.

6. The antibody screening kit of claim 5, wherein the step a) of producing G protein-coupled receptors (GPCRs) in E. coli cells comprises the steps of:
a1) culturing the E. coli transformed with G protein-coupled receptors (GPCRs);
a2) overexpressing the G protein-coupled receptors (GPCRs);
a3) lysing the E. coli to release the G protein-coupled receptors (GPCRs) outside the cell; and
a4) solubilizing and purifying the G protein-coupled receptors (GPCRs).

7. The antibody screening kit of claim 5, wherein the lipids are selected from 1,2-dipalmitoyl-sn-glycero-3-phosphocholine (DPPC), 1,2-disteroylsn-glycero-3-phosphocholine (DSPC), L-a-phosphatidylcholine (HSPC), 1-palmitoyl-2-glutaroyl-sn-glycero-3-phosphocholine (PGPC), 1,2-dilauroyl-sn-glycero-3-phosphocholine (DLPC), 1,2-dimyristoyl-sn-glycero-3-phosphocholine (DMPC), 1,2-dioleyl-sn-glycero-3-phosphocholine (DOPC), and a mixture thereof.

8. The antibody screening kit of claim 5, wherein the membrane scaffold protein in step b) is MSP1E3D1.

9. The antibody screening kit of claim 5, wherein, in step c), the G protein-coupled receptors are mixed at a molar ratio of 1:2 to 1:30 with the membrane scaffold proteins and at a molar ratio of 1:200 to 1:2500 with the lipids.

10. The antibody screening kit of claim 1, wherein the nanodisc ranges in diameter from 10 to 25 nm.

11. The antibody screening kit of claim 1, wherein the nanodisc has a structure in which one type of G protein-coupled receptors (GPCRs) is embedded into a single nanodisc.

12. A method for screening an antibody drug, using the antibody screening kit of claim 1.
